(19) United States
(12) Patent Application Publication
(10) Pub. No.: US 2016/0193048 A1
(43) Pub. Date: Jul. 7, 2016

(54) ULTRASOUND-COMPATIBLE ARTIFICIAL CRANIAL OPERCULUM

(71) Applicant: Francesco Ugo PRADA, Milano (IT)

(72) Inventor: Francesco Ugo PRADA, MILANO (IT)

(21) Appl. No.: 14/915,972

(22) PCT Filed: Sep. 4, 2014
(86) PCT No.: PCT/EP2014/068837

§ 371 Date: Mar. 2, 2016

(30) Foreign Application Priority Data
Sep. 5, 2013 (IT) MI2013A001453

(51) Int. Cl. A61F 2/28; A61L 27/16

(52) U.S. Cl. CPC A61F 2/2875; A61L 27/16; A61L 2430/02

(57) ABSTRACT

What is disclosed is an artificial cranial operculum (1) to replace a bone operculum (2) removed from a cranial theca (7) during a neurosurgical procedure that involves a craniotomy. The artificial cranial operculum (1) is made of material that is rigid, biocompatible, sterilizable, and compatible with ultrasound and with nuclear magnetic resonance. Preferably the artificial operculum is made of polymethylpentene.

The present invention relates to an ultrasound-compatible artificial cranial operculum.

Gliomas are the most common primitive tumors of the central nervous system. 70% of them are malignant (WHO grade III anaplastic astrocytoma, WHO grade IV glioblastoma), and patients who are affected by them on average survive for approximately 18 months for anaplastic astrocytoma and less than a year for glioblastoma. Such tumors are treated by surgical removal, as well as the administration of adjuvant treatments such as chemotherapy and radiotherapy. Such approach, however, has had and still has a low success rate in reducing mortality; surgery is effective in containing the mass effect that is due to the presence of the tumor, but it is not curative owing to microscopic invasiveness and local recidiva. Consider that the average survival time after surgical resection alone is six months, with only 7% of patients surviving longer than two years.

The use of imaging methods, i.e. of techniques to obtain images that provide diagnostic information, now plays an essential role in the carrying out of neurosurgical procedures, making it possible to best plan the procedure and enabling the anatomical and functional definition of the lesion. Furthermore, imaging methods can help the orientation of the neurosurgeon/neuro-oncologist during the resection. For example, the intraoperative use of ultrasound in neurosurgery, by placing the ultrasound probe directly on the brain, enables an excellent definition of the cerebral lesions.

The use of imaging methods continues in the immediate post-operative phase, in order to evaluate the extent of the resection of the lesion and of the efficacy of adjuvant therapies. Such therapies can however have marked side effects, as is the case with radiotherapy, or high costs, as is the case with chemotherapy. Furthermore, some patients may not respond to a form of chemotherapy and therefore it is necessary to identify such cases and apply a different treatment. Early identification of these patients, in addition to improving their treatment, would result in a considerable economic saving.

Although ultrasound is a widely used tool in the field of general diagnostic radiology, it is limited to only a few areas in cerebral diagnostics. In fact, in post-operative (follow-up) checkups, the highly hyperechogenic nature of the cranial theca prevents ultrasound from penetrating into the cranial cavity, with the exception of the ocular and temporal acoustic fenestra. Repositioning the bone operculum, removed following the neurosurgical procedure, in fact constitutes a barrier to ultrasound and does not allow follow-up checkups of the patient using ultrasound.

The same occurs when the cranial theca is reconstructed according to prior art solutions. For instance, US2006/224242 discloses an implant for reconstruction of craniofacial defects which uses a composite structure comprised of a surgical grade metal provided in a planar or curved sheet form that is encased within a malleable biocompatible material, such as a polyolefin, in particular high density polyethylene.

Although occasionally available, the ultrasound methods used to get past the cranial theca still do not enable an accurate and definite evaluation of the brain parenchyma.

The aim of the present invention is to enable an effective use of ultrasound in cerebral diagnostics, by making it possible to perform post-operative serial checkups in order to monitor the progress of the illness, and furthermore in order to administer therapies using an ultrasound release system.

In accordance with the invention such aim is achieved by way of an artificial cranial operculum to replace a bone operculum removed from a cranial theca during a neurosurgical procedure which comprises a craniotomy, said artificial cranial operculum being characterized in that it is made of a material that is rigid, biocompatible, sterilizable, and compatible with ultrasound and with nuclear magnetic resonance,

The characteristics of the present invention will be made clear by the following detailed description of an embodiment thereof, which is illustrated by way of non-limiting example in the accompanying drawings, wherein:

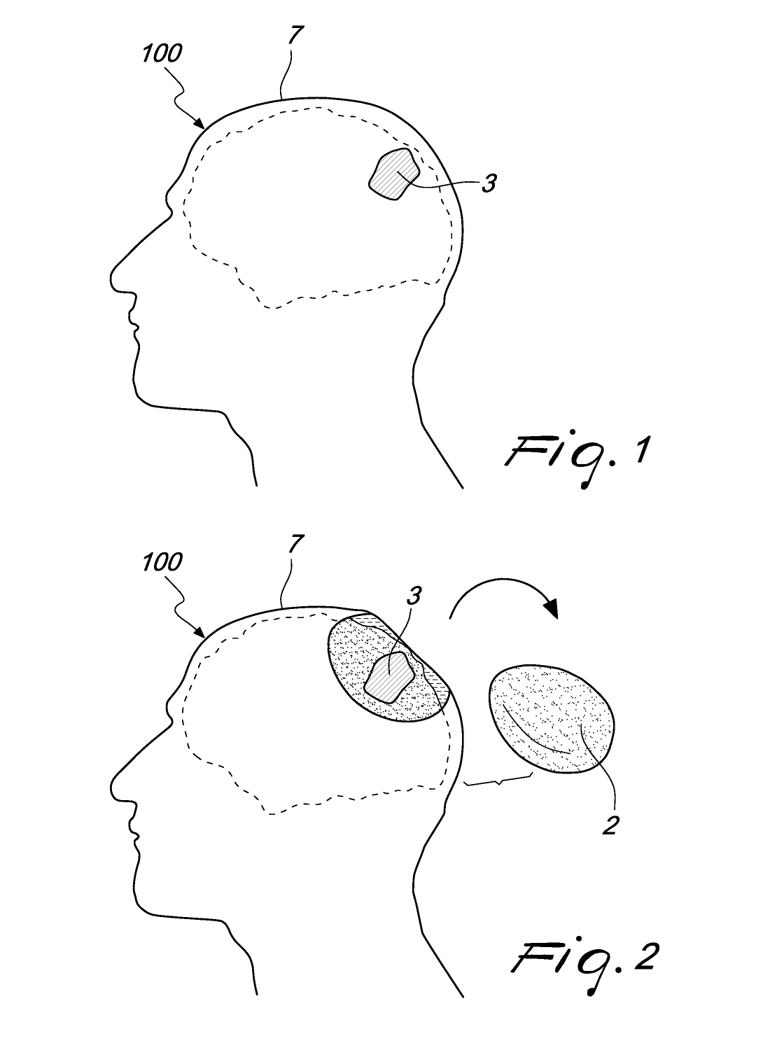

FIG. 1 is a schematic side view of a patient affected by a high-grade glioma;

FIG. 2 is a schematic side view of a patient affected by a high-grade glioma in a first step of a neurosurgical procedure in which a cranial operculum is removed from the cranial theca;

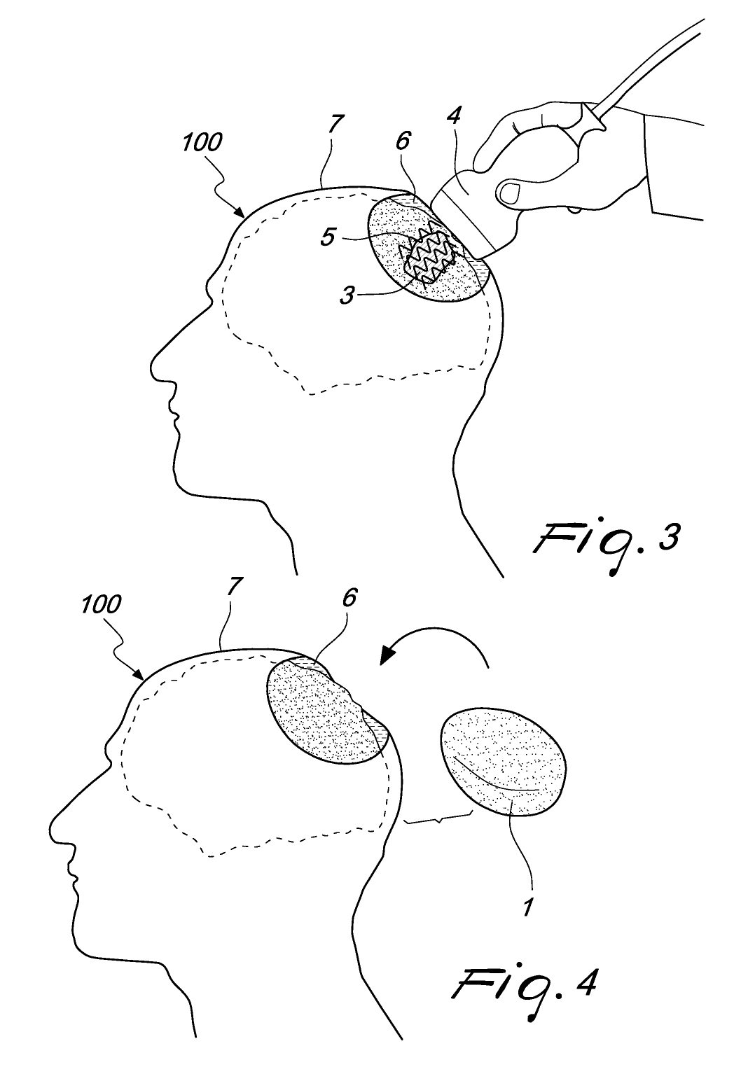

FIG. 3 is a schematic side view of a patient affected by a high-grade glioma in a second step in which an ultrasound probe is placed directly on the brain tissue;

FIG. 4 is a schematic side view of a patient affected by a high-grade glioma in a third step in which, with the glioma surgically removed, an artificial cranial operculum according to the present invention is being positioned on the cranial theca;

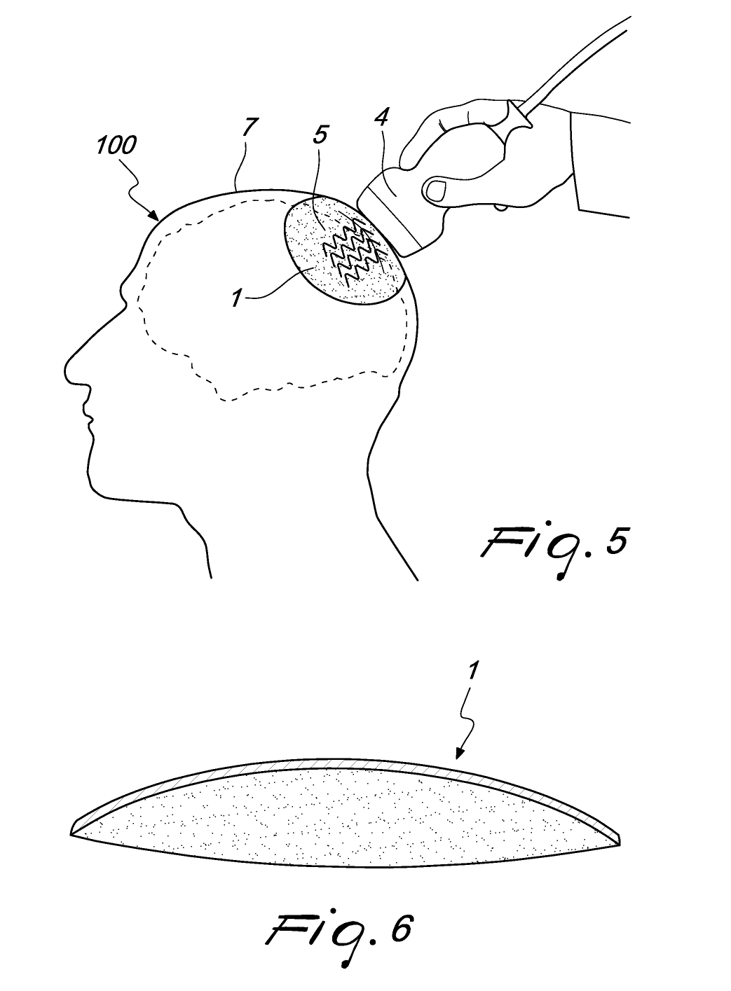

FIG. 5 is a schematic side view of a patient with the glioma removed, in which the ultrasound probe is placed on the artificial cranial operculum in FIG. 4, which in turn is placed on the cranial theca of the operated patient;

FIG. 6 is a schematic view of an artificial cranial operculum according to the present invention.

The artificial cranial operculum 1 according to the present invention is an element that is used in a specific step of a neurosurgical procedure. In the case under discussion, the procedure is adapted to resection a primitive tumor of the nervous system (glioma), shown schematically in FIGS. 1, 2 and 3. Obviously the tumor can be of different size and shape and may be differently positioned.

The artificial cranial operculum 1 is constituted by a cap made of rigid material which will be used to substitute a bone operculum 2 that has been removed following a craniotomy, i.e. as a consequence of the procedure that enables access to the inside of the cranial theca 7 of a patient 100 affected by high-grade glioma 3, in order to execute the removal of same. For this reason the artificial cranial operculum 1 must be an exact replica of the native bone operculum 2 so as to perfectly fit with the rest of the cranial theca 7. So that the artificial operculum 1 will be metabolized with no damaging effects for the patient 100, it is necessary that it be made of a material that is biocompatible and which is also sterilizable before application.

The artificial operculum 1 can have holes (not shown in the figures) that are adapted to secure it to the cranial theca 7 by way of suture thread.

At the end of the neurosurgical procedure to remove the tumor, in order to prevent the repositioning of the native bone operculum 2 from impeding the follow-up checkups of the patient with ultrasound techniques, the artificial cranial operculum 1, which is intended to substitute the removed bone operculum 2, is made of a material that is compatible with ultrasound, i.e. of a material that offers no resistance to the passage of ultrasound. Furthermore, in order to also enable the post-operative use of methods that require nuclear magnetic resonance (NMR), the artificial cranial operculum 1 is also NMR-compatible.

Preferably, the operculum is made of polymethylpentene (TPX), a material used in a wide variety of medical applications owing to its low impedance, similar to that of organic fabrics.

The implementation and application of the cranial operculum 1 during a surgical operation occurs in the following manner (FIG. 1-5).

In a preliminary step of the neurosurgical procedure, a study is conducted on the patient 100 hit by glioma 3 by way of computerized tomography (CT) and nuclear magnetic resonance (NMR), which enables the anatomical and functional definition of the tumor 3.

Then surgical planning of the craniotomy is done with neuronavigation. On the basis of such planning, the region and shape of the craniotomy are decided, and the desired cranial operculum 1 is designed on the images extrapolated from the CT.

The images are transferred to a 3D CAD package with “mirroring” of the native bone 2. In this way as 3D model is built on the basis of which the artificial cranial operculum 1 will be produced.

Then an artificial cranial operculum 1 is built of polymethylpentene (TPX), on the basis of the 3D model, and this is sterilized.

Now all is ready for executing the first step of the previously-planned craniotomy surgery (FIG. 2), under the guidance of neuronavigation.

In a second step, during the procedure, the ultrasound probe 4 is placed directly on the brain tissue (FIG. 3). The ultrasound 5 passes through the open space 6 of the bone cranial operculum, thus enabling the cerebral lesion 3 to be defined.

In a third step (FIG. 4), after the removal of the tumor, the artificial cranial operculum 1 is repositioned and is fixed by means of holes and suture thread (not shown in the figure).

Ultrasound 5 is capable of passing through the cranial operculum 1 (FIG. 5) made of polymethylpentene (TPX) and make it possible to view the brain during follow-up checkups of the patient.

The creation of the ultrasound-compatible artificial cranial operculum 1 in substitution of the bone operculum 2 of the patient 100 who has been operated on for high-grade glioma 3 directly enables the attending medical practitioner (neurosurgeon/neuro-oncologist) to perform serial ultrasound checkups to monitor the progress of the illness and also to administer locoregional therapies using an ultrasound release system. This translates to a simplification of the investigations, in that instruments for chemotherapy or nuclear magnetic resonance are not required, and furthermore it represents a saving of money, time and human resources.

In particular, the use of the artificial cranial operculum according to the present invention enables the use of the ultrasound technique combined with the CEUS (Contrast Enhanced UltraSound) method, recently introduced, which makes it possible to identify neoplastic lesions with ultrasound contrast means which consist of micro-bubbles of air or inert gases encapsulated in a proteic layer or a layer of polymers. The micro-bubbles typically have an average diameter similar to that of red corpuscles and can be carried in blood capillaries and through the lungs. They inherently produce a strong ultrasound signal owing to the ample acoustic impedance generated by the gas/blood interface, and this signal is further boosted because the micro-bubbles themselves, struck by the ultrasound, echo at specific frequencies, as a function of their diameter, producing an ultrasound signal, as well as reflecting it. Such methodology, which is simple in technical and organizational terms, makes it possible to effectively evaluate the characteristics of the brain tumoral lesions and to identify the tumoral residues during removal of the lesion, thus enabling greater radicality.

The invention, thus conceived, is susceptible of numerous modifications and variations, all of which are within the scope of the appended claims.

Moreover, all the details may be substituted by other, technically equivalent elements.

In practice the materials employed, provided they are compatible with the specific use, and the contingent dimensions and shapes, may be any according to requirements and to the state of the art.

The disclosures in Italian Patent Application No. MI2013A001453 from which this application claims priority are incorporated herein by reference.

Where technical features mentioned in any claim are followed by reference signs, such reference signs have been inserted for the sole purpose of increasing the intelligibility of the claims and accordingly such reference signs do not have any limiting effect on the interpretation of each element identified by way of example by such reference signs.

1. An artificial cranial operculum (1) to replace a bone operculum (2) removed from a cranial theca (7) during a neurosurgical procedure which comprises a craniotomy, said artificial cranial operculum (1) being characterized in that it is made of a material that is rigid, biocompatible, sterilizable, and compatible with ultrasound and with nuclear magnetic resonance.

2. The artificial cranial operculum (1) according to claim 1, characterized in that it is made of polymethylpentene (TPX).

3. The artificial cranial operculum (1) according to claim 1, characterized in that it comprises holes suitable for securing it to the cranial theca (7) by way of suture thread.

4. A method of implementation and application of the artificial cranial operculum (1) during a neurosurgical procedure, characterized in that it comprises the conducting, on a patient (100) affected by glioma (3), of a study by way of computerized tomography (CT) and nuclear magnetic resonance (NMR), the surgical planning of the craniotomy with neuronavigation and design of the desired cranial operculum (1), the construction of a 3D model of the desired cranial operculum (1), the production of an artificial cranial operculum (1) on the basis of said 3D model and the sterilization thereof, the execution of the craniotomy surgery with removal of a bone operculum (2), the positioning of an ultrasound probe (4) directly on the brain tissue and, after removal of the tumor, the positioning of the artificial cranial operculum (1) in place of the removed bone operculum.
